(19) United States
(12) Patent Application Publication
(10) Pub. No.: US 2021/0171887 A1
(43) Pub. Date: Jun. 10, 2021

(54) APPARATUS AND METHOD FOR LEVITATIONAL BIOFABRICATION OF ORGAN AND TISSUE ENGINEERED CONSTRUCTS USING TISSUE SPHEROIDS AND MAGNETOACOUSTIC BIFIELD

(71) Applicant: VIVAX BIO, LLC, New York, NY (US)

(72) Inventors: Vladislav PARFENOV, Podolsk (RU); Yusef KHESUANI, Moscow (RU); Vladimir MIRONOV, Moscow (RU); Frederico David ALENCAR DE SENA PEREIRA, Vitória da Conquista (BR); Elena BULANOVA, Moscow (RU); Elizaveta KUDAN, Moscow (RU); Stanislav PETROV, Pushkino (RU); Alexander OSTROVSKIY, Moscow (RU); Yakov BALAKHOVSKY, New York, NY (US); Oleg SAPOZHNIKOV, Moscow (RU); Vera KHOKHLOVA, Moscow (RU); Sergey TSYSAR, Moscow (RU); Victor SVET, Moscow (RU)

(21) Appl. No.: 17/094,350

(22) Filed: Nov. 10, 2020

Related U.S. Application Data
(60) Provisional application No. 62/933,641, filed on Nov. 11, 2019.

(51) Int. Cl. C12M 3/00; C12M 1/12; C12M 1/26; C12M 1/42; G01R 33/12

(52) U.S. Cl. CPC C12M 21/08; C12M 25/14; G01R 33/1269; C12M 35/06; G01R 33/1261; C12M 33/00

(57) ABSTRACT

This invention is related to technology of tissue-engineered constructs biofabrication from tissue spheroids. This novel technology of scaffold-free, nozzle-free and label-free bioassembly opens a unique opportunity for rapid biofabrication of 3D tissue and organ constructs with complex geometry. A combination of intense magnetic and acoustic fields could enable rapid levitational bioassembly of complex-shaped 3D tissue constructs from tissue spheroids at low concentration of paramagnetic agent (gadolinium salt) in the medium. Magnetic field provides objects levitation due to field configuration with the lowest magnetic field density in the center of working volume of medium with tissue spheroids, and three-dimensional acoustic field forms internal and external construct geometry by means of acoustic radiation forces.

### FIELD OF INVENTION

This invention is related to technology of tissue engineered constructs biofabrication from tissue spheroids, in particular, to apparatus and method for scaffold-free, nozzle-free and label-free printing of 3D tissue and organ constructs with complex geometry with inner channels and based on manipulation of spheroids in working area by means of a hybrid combination of magnetic and acoustic fields.

### BACKGROUND OF THE INVENTION

One of the biofabrication ultimate targets consists in the creating of three-dimensional tissue constructions exercising the same functions as human tissues and organs. The creation of such three-dimensional constructions would allow using them for medicinal preparations and medical devices testing, furthermore, it would allow creating organ structures exercising human organs' functions. Since it is commonly known that the deficit of the organs donated for transplantation constitutes a real problem, the biofabrication of organ constructions would become a solution of this global clinical issue.

Tissue spheroids are finding ever-widening application as building blocks for tissue and organ constructs fabrication [1]. This is because tissue spheroids have some advantages as compared to individual cells.

By designing tubular organs the main problem consists in the difficulty to obtain a complex geometry imitating natural tissues structure. This is especially the case when forming microscale cell layers arrangement and producing extracellular matrix which are necessary for reconstruction of complex anatomical architecture of the network of tubular organs having numerous bifurcations or physiological thickenings in maximum hydrostatic pressure areas [2].

Current additive technologies provide for the use of different types of dispensers (nozzles) for layer-by-layer application of biomaterials, and the retention of such tissue requires the use of temporary supporting structures (scaffolds) such as hydrogels, polymers, metal rods, etc. to retain spheroids in certain spatial positions. However, the use of such supporting structures attended by some inherent disadvantages including, in particular, increase of printing process duration, impossibility of spheroids arrangement in close contact with each other and absence of the methods allowing bioprinting to create a network of branching channels within tissue engineered constructs.

As an alternative to scaffold -based approach, new scaffold-free technologies were developed based on the use of physical fields as a temporary support for quick fabrication of tissue constructions with complex geometry.

Several research teams successfully applied acoustic waves as a tool for formation of cells and tissue spheroids pattern in closely packed functional three-dimensional tissue constructions such as three-dimensional structures of heart tissue [3], three-dimensional circular tissue constructions generated by fusion of fibroblasts and endothelial cell spheroids [4] and three-dimensional circular soft cellular robots made of neurons and astrocytes [5].

Acoustic levitation methods for three-dimensional objects generation are also known from publications U.S. Pat. No. 10,695,980 B2, IN201811047150 A, WO2019078639 A1, KR102140967B1.

Acoustic (ultrasonic) field may be used for tissue spheroids levitation. Due to a strong dependence of the acoustic field on ultrasonic source shape and size, its operating frequency and boundary conditions it is possible to create complex three-dimensional acoustic traps. When tissue spheroids are placed in the volume exposed to ultrasonic field, so called “acoustic radiation force” is created being the result of pulse transmission from acoustic wave to absorbing or scattering objects. Radiation force amplitude and direction depend on certain field structure. For example, if the acoustic field is used in the form of standing wave, and wavelength exceeds spheroids diameter, the resulting radiation force will move spheroids to acoustic pressure nodes.

However, the ultrasonic field is more suitable for creation of small acoustic traps and doesn't allow retaining large volume of spheroids which imposes limitation on creation of complex three-dimensional objects. Manipulations with large size constructs with the acoustic field only will require high radiation power leading to destruction of such construct.

Besides the acoustic field, the magnetic levitation principle may also be used as a tool for complex three-dimensional tissue structures creation. Magnetic-levitation assembly, in its turn, may be obtained by using paramagnetic agents [6-9] or magnetic nanoparticles captured by cells [11-12]. It should be noted that magnetic nanoparticles (marks) are used in potentially toxic concentrations to reach sufficiently high magnetic force which significantly confines the scope of this method. In contrast to nanoparticles, paramagnetic agents can be easily removed from constructions upon biotechnology process completion. However, high concentrations of gadolinium salts usually used as paramagnetic agent for magnetic levitation assembly may also be toxic for cells [13, 14].

In recent times, biofabrication methods are being developed in which three-dimensional tissue constructions are assembled by magnetic levitation method in non-toxic paramagnetic liquid without the use of substrates or frameworks as well as without the use of magnetic marks [9]. Tissue spheroids gathered together in magnetic trap contact each other and thereby fuse while forming a three-dimensional tissue construct.

There's also known a three-dimensional object assembly in zero-gravity environment using magnetic levitation (U.S. Pat. No. 9,908,288 B2). However, magnetic levitation allows fabrication of simple shape structures only while real organs contain hollow blood vessels. Therefore, an important task is to form tissue constructions with internal channels.

One of the simplest and widespread shapes in tissues and organs is a circle or a tube since it is the shape blood vessels and capillary tubes have. Vascularization problem is sensitive for bioengineering since it is necessary for tissue viability maintenance not only to assemble a structure from cells but also to feed it, and nutrients penetration depth through cellular medium does not exceed 0.1 mm which defines maximum radius of viable spheroids.

Therefore, the state of the art does not allow performing quick levitation assembly of complex shape construction from tissue spheroids randomly distributed in working volume of nutrient medium which would exercise functions of blood vessels, for example, and would have functional activity and viability. The proposed method of hybrid magnetoacoustic levitation bioassembly is aimed at breaking of existing technology barriers. In particular, spatial distribution of biological objects may be only defined by physical fields applied and, in comparison to conventional biotechnology methods, does not depend on physical and chemical properties of biomaterials included in frameworks or “bioink” compound.

### DISCLOSURE OF INVENTION

The objective of the present invention is to develop a method for creation of biotechnological construction of viable and contractile complex shape tissue including inner channels from spheroids randomly distributed in working volume of nutrient medium without the use of substrates, frameworks (scaffolds), magnetic marks (labels) and toxic medium.

In order to perform this task a newly developed biotechnology is proposed: hybrid magneto-acoustic levitation bioassembly of functional human tissues.

The magnetic field retains objects not affecting cell material viability but affecting geometry of created construct, and acoustic field forms a complex geometry of created construct. In proposed technical solution the use of two physical field types produces an unexpected synergetic effect, since it allows not just to assemble objects in acoustic wave nodes or in areas with the lowest magnetic field density, but to affect a solid construct thus forming inner cavities in the areas where acoustic field radiation force exceeds magnetic force.

It is obvious that the degree of complexity of created three-dimensional objects geometry depends on the complexity of acoustic field being applied which, in its turn, may be created using a large number of radiating elements.

In order to develop the said approach the following technological steps were performed: i) development of hardware providing magnetoacoustic levitation bioassembly, ii) performance of mathematical simulation and implementation of forming biotechnological fabrication of complex biological structures such as rings or tubes, and iii) functionality testing of biotechnological 3D tissue tubular construction.

The method and the apparatus described below allow performing quick scaffold-free, label-free, and nozzle-free biofabrication of organ constructs using tissue spheroids by means of specially created nonuniform magnetoacoustic field (“bifield”). Such “bifield” acts as a temporary physical scaffold. Furthermore, the method and the apparatus described below may be used for biofabrication from not only tissue spheroids but also from suspensions of separate cells of different types (muscular tissue cells, epithelial cells, etc.) as well as for non-organic materials.

Method

The proposed method may be described as a fast levitation assembly of constructs in liquid growth medium (nutrient medium) containing randomly distributed tissue spheroids. Basic tool used for assembly is a combination of non-uniform magnetic field and standing wave acoustic field actioning on test tube with the medium creating magnetoacoustic trap in certain area. Magnetic field is convenient for retaining large volume of spheroids but it has limited capabilities for thin structure formation. Ultrasonic field, in contrast, is more suitable for small traps formation. The combination of magnetic and acoustic fields opens up new opportunities and allows creation of complex three-dimensional structures directly in nutrient medium.

Spheroids floating in nutrient medium are concentrated in magnetoacoustic trap area due to combined action of gravitational, magnetic and acoustic forces. In such case, gravitational forces are compensated by magnetic forces in vertical direction and tissue spheroids move towards each other due to magnetic gradient in horizontal plane while being lifted over test tube bottom. As a result, tissue construct assembly takes place when spheroids fuse each other in nutrient medium without contacting the substrate. Construction shape may be changed by acoustic radiation force application. The acoustic field may have a complex structure which depends on source geometry, wave frequency and boundary conditions in exposure area. Therefore, construct shape depends on configuration of the acoustic field applied thereto.

Magnetic force appears due to spatial non-uniformity of the magnetic field and difference in magnetic susceptibility between spheroids and nutrient medium. The more magnetic susceptibility difference present, the more is the magnetic force. That is the reason why gadolinium salts solution shall be added to medium for effect intensification. Thus, liquid medium becomes paramagnetic relative to assembly objects and spheroids become diamagnetic as the water.

Constructs created in such a way may have spheroidal, toroidal, ellipsoidal or other shape defined by the shape of chosen magnetoacoustic “bifield”—certain configuration of magnetic and acoustic fields.

After formation of construct assembly of the chosen shape a possibility to form divided channels network therein emerges. In order to achieve this it is necessary to apply a non-uniform acoustic field in addition to a non-uniform magnetic field. The non-uniform acoustic field is a specially selected combination of standing or running ultrasonic waves propagating from one or several projectors located relatively close to each other in a certain manner.

If running ultrasonic wave strike a barrier it is exposed to both variable and constant pressure. Medium concentration and exhaustion areas appearing during passing of ultrasonic waves create additional pressure changes in the medium in relation to surrounding external pressure. Such additional external pressure is called radiation pressure (force).

Radiation force is a parameter depending on spatial acoustic energy density variation in propagating wave. Such energy density variation may be caused by non-uniformity of medium acoustic characteristics. Projectors inclination angle influences the direction of generated radiation pressure. Inclination angle is chosen in accordance with the required barrier movement trajectory.

Such continuously acting acoustic force works for creation of the field within the assembled construct causing formation of one or several branching channels whose size, shape and complexity are defined by the number of acoustic projectors and their spatial arrangement. Channels are developed beforehand by means of a three-dimensional computer simulation of acoustic fields which leads to the values of amplitudes, frequencies and phase shifts for each particular acoustic transducer located in specific location outside of the working area. In order to facilitate the calculation of complex acoustic fields it is possible to use learning neural networks. In one embodiment of the method according to the present invention it is possible to design all channels of a created tissue construct simultaneously by spatial arrangement of acoustic transducers and scheduling their radiation characteristics. In one embodiment of the method according to the present invention a sequential design of each tissue construct channel and a sequential change of created acoustic trap configuration in non-uniform magnetic field are possible.

The basic concept of vascularization approach consists in construct assembly formation with subsequent formation of its internal particularities.

Channels are formed if the acoustic field acting on tissue spheroids is stronger than the vector sum of all other fields affecting spheroids in this area. While the magnetic field works for tissue spheroids packing with maximum density, the acoustic field in certain areas within the constructs separates them by creating inner channels. If the magnetic field is stronger than the acoustic field radiation force, channels are not formed.

For example, this method allows creating biotissue engineered constructs with a network of divided channels having a diameter of 300 to 900 μm within approximately 60 seconds. After biofabrication the levitating construct is exposed to the fields till the completion of tissue spheroids fusion process which usually lasts for about 24 hours. Fusion time depends on chosen cells type from which the spheroids were made and, thus, may vary within certain limits.

The scheme of experiment on levitation magnetoacoustic biofabrication of construct from tissue spheroids is shown in FIG. 1.

Fields shall be switched on in accordance with the chosen shape of the object received. Magnetic to acoustic field ratio is defined by their influence on spheroids: magnetic forces compensate gravitation force and assemble tissue spheroids in the center of “magnetic hole” and acoustic radiation force is the force of pressure on tissue spheroids, therefore, it moves them in a space due to pressure. These fields do not influence on each other, they affect spheroids and the parameters of the fields shall be selected through this influence.

Magnetoacoustic assembly method may be conditionally divided into several stages:
1. Process of construct creation from spheroids—tissue spheroids assembly in a predetermined shape defined by magnetic field parameters and acoustic field structure. If fields are switched off immediately after assembly the shape falls down.
2. Supporting stage—tissue spheroids retention in assembly shape. This stage starts immediately after assembly process completion. Supporting stage lasts for 8 to 24 hours, and ultrasonic wave intensity shall be sufficiently low to avoid tissue spheroids damage during a long-term exposure.
3. Spheroids fusion process is the process where spheroids already interact, and the shape is retained after field switching off. The time of tissue construct creation is defined by the requirement to fusion stage. Continuous tissue formation requires from 20 to 72 hours.

Therefore, the magnetoacoustic biofabrication method may be considered as further extension of “scaffield” concept where only physical fields are used for temporary support of cells or their aggregates. Furthermore, such approach allows to avoid using natural and synthetic biomaterials whose clinical application still involves the risks of immunologic rejection development or imperfect resorption often leading to the development of inflammatory responses or fibrosis in recipient's organism.

Apparatus

The method includes creation of non-uniform magnetoacoustic “bifield” and placement of tissue spheroids randomly distributed in liquid in a working area.

The non-uniform magnetic field is generated using several permanent magnets of a defined shape located relative to each other in a certain manner (FIG. 2). Neodymium magnets may be used for this purpose [8]. Higher magnetic field gradients and densities and, therefore, larger working areas may be achieved using Bitter magnets or superconducting magnets.

In order to achieve the highest magnetic field gradient, the apparatus consists in one of the embodiments of two oppositely oriented circular magnets with a hollow space between them. At least one ultrasonic transducer and medium with tissue spheroids are placed therein.

Permanent magnets or electric magnets are arranged in the manner allowing to create local microgravitation area where the effect of all forces on diamagnetic objects (tissue spheroids) in nutrient medium (paramagnetic medium) is compensated.

In the proposed apparatus upward-directed Archimedes' force acting on tissue spheroids in growth medium, downward-directed force of gravity and magnetic force directed to local minimum of the magnetic field make a simultaneous impact on the object in static conditions. Magnetic force moment appears only if the magnetic field is not uniform. Then the effective magnetic force acting on the object in non-uniform magnetic field will be equal to:

F=X m/2∇(B²),

where

X—specific magnetic susceptibility of substance (for paramagnetic substances X>0, and for diamagnetic substances X<0), m—particle mass, B—magnetic field induction. The sign of the obtained value defines the direction of magnetic force action.

As a result, diamagnetic objects (tissue spheroids) will be ejected to the area with a lower field density (magnetic trap) under the action of the magnetic force. In the Earth gravitation conditions the equilibrium point is located at a defined distance from local minimum of the magnetic field while in microgravitation medium conditions the assembly point coincides with the magnetic field minimum area. In order to provide levitation in the Earth gravitation conditions, the magnetic field gradient in the gravity force direction shall be at least 1.3 T/cm and the growth medium shall contain paramagnetic metal salts such as gadolinium salts with Gd³⁺ concentration equal to 50 mM. The scheme of the arrangement of magnets located with analogous poles facing each other and horizontal and vertical magnetic field profiles are shown in FIG. 2.

The acoustic field is created by ultrasonic projectors with several outputs located outside of the magnetic trap. Acoustic radiation comes to the magnetic trap from different directions which can, for example, coincide with X, Y and Z axes. Due to creation of longitudinal standing acoustic waves in three dimensions and controllability of amplitudes and phases of signals fed to radiation sources, complexly distributed zones with the required acoustic pressure levels in the form of divided channels can be obtained. (FIG. 3).

Acoustic radiation sources for bifield generation may be placed, for example, in a gap between magnet-like poles. Apparatus for magnetoacoustic “bifield” generation is shown in FIG. 4: a) spheroids in initial moment and after construct assembly; b) apparatus for creation.

The apparatus also includes at least one video-camera connected with data processing device including at least one data display and at least one microprocessor. Herewith the microprocessor makes it possible to control characteristics of ultrasonic projectors generating the acoustic field.

Data transfer means are selected from among the devices intended for implementation of the communication process between different devices by means of wire and/or wireless communication, such as: GPS modem, BLE module or Bluetooth, Wi-Fi transceiver, etc.

The proposed apparatus and the method do not impose any limitations on size, complexity and quality of tissue or organ constructs being constructed which are defined directly by the power of magnetic field generator and the number of acoustic radiation sources placed on acoustic arrays.

### BRIEF DESCRIPTION OF FIGURES

The attached drawings included in this description and constituting an integral part hereof show the embodiment of the invention and, in connection with the above summary description of the invention and the embodiment description detailed below serve for explanation of the principles of the present invention.

FIG. 1. Scheme of experiment on levitation magnetoacoustic fabrication of construct from tissue spheroids.

FIG. 2. Non-uniform magnetic field created between two magnets with analogous poles facing each other.

FIG. 3. Areas where the acoustic field generated by the set of acoustic projectors is stronger than the magnetic field during hybrid magnetoacoustic fabrication of construct from tissue spheroids.

FIG. 4. Assembly and formation (biofabrication) of organ constructs under the action of magnetic and acoustic fields: a) spheroids in initial moment and after construct assembly; b) apparatus for creation.

FIG. 5. Example 1. Biofabrication of circular construct from tissue spheroids in magnetoacoustic field. (a) design and (b) image of magnetoacoustic apparatus with cylindrical ultrasonic transducer. Force lines show magnetophoretic forces direction, the area within piezoelectric transducer shows distribution of acoustic pressure amplitude; (c) results of numerical simulation of spheroids assembly in the magnetoacoustic field. Background color shows Gorkov's potential amplitude; (d) experimental distribution of tissue spheroids in the magnetoacoustic field, second circle formation. Inner circle diameter is 1.5 mm; (e, f) tissue spheroids fusion in a circle within 18 hours. Circle diameter is 1.5 mm.

FIG. 6. Spheroids circle formation in the beginning of the experiment and after 20 hours of fusion from (a, b) smooth muscle cells and (c, d) chondrocytes.

FIG. 7. Optical scheme used for construction assembly registration in magnetoacoustic environment.

FIG. 8. Design of cuvette with acoustic transducers: a—cuvette layout; b—cuvette model with spatially spread elements; c—cuvette with liquid; d—3D model of agarous cuvette with tissue spheroids inside; e—agarous cuvette inside a cylindrical transducer; f—cuvette installation in Bitter magnet; g—Bitter magnet used in the experiments (side view); h—frequency dependence of transducer electric power at ideal load of 50 Ohm. Profile peaks conform to resonance frequencies. Particles assembly was performed near these frequencies.

FIG. 9. Construct assembly stages: using magnetic levitation, acoustic field from circular piezoelectric transducer and standing acoustic field from cylindrical piezoelectric transducer.

FIG. 10. Biofabrication of the construction from tissue spheroids at different acoustic field parameters: a—process of circle assembly in levitation state using acoustic and magnetic fields; b—levitating tubular construction; c—construction diameter change depends on frequency; d—convergence of experimental and levitation diameters by frequency dependence.

FIG. 11. Results of numerical simulation of the acoustic permanent field and the construction assembly process in magnetoacoustic field. a—acoustic pressure amplitude distribution within a transducer; b—radiation force distribution by magnitude; c—illustration of particles concentration in acoustic pressure field nodes; d—coaxial structure of standing wave within cylindrical transducer; e—resulting tubular node obtained in magnetoacoustic field as a result of particles movement monitoring.

FIG. 12. Characteristic of tubular construction fabricated by means of the magnetoacoustic levitation method during 8 hours with 20 mM of gadobutrol: a—photograph of construct inside agarous cuvette; b—stereo image of the construct; c—construct histology; d—construct's live/dead analysis: phase contrast, calcein-AM and propidium iodide from left to right; e—construct SEM; f—construct contraction during 120 minutes in the presence of 50 nM of endothelin-1; g—dynamics of area reduction caused by 50 nM of endothelin-1.

FIG. 13. Influence of different gadobutrol concentrations on spheroids' tissues fusion. Time curves of intersphere angles (a), contact length (b) and doublet length (c) during fusion.

FIG. 14. Biofabrication of tissue spheroids for tubular constructions assembly. Tissue spheroids distribution by diameter (a) and roundness (b), n=324. Influence of different gadobutrol concentrations on viability (c) and mechanical properties (d) of tissue spheroids.

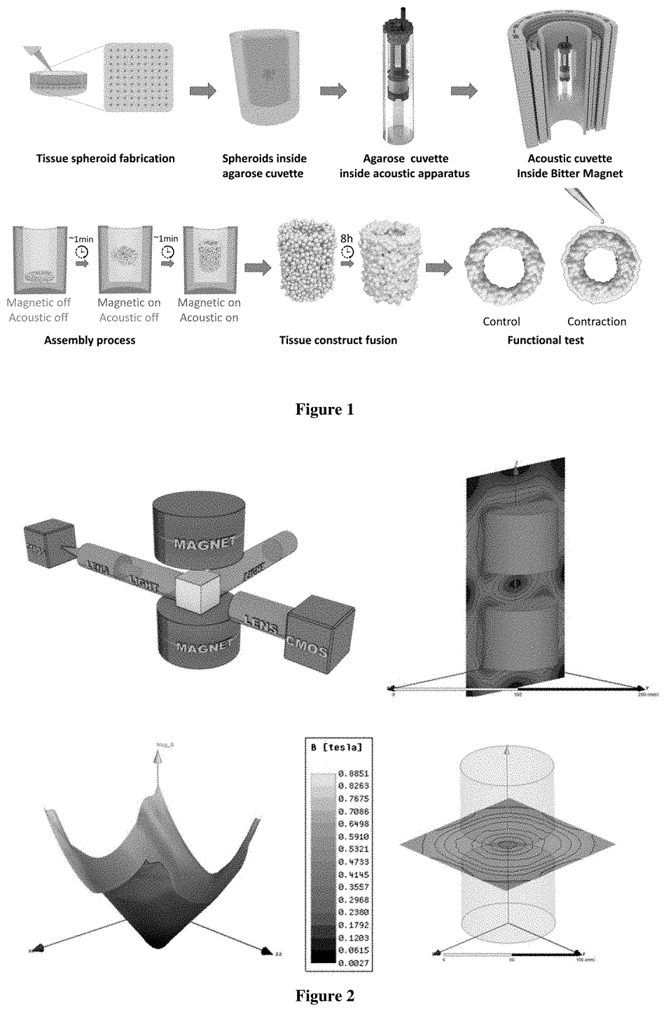

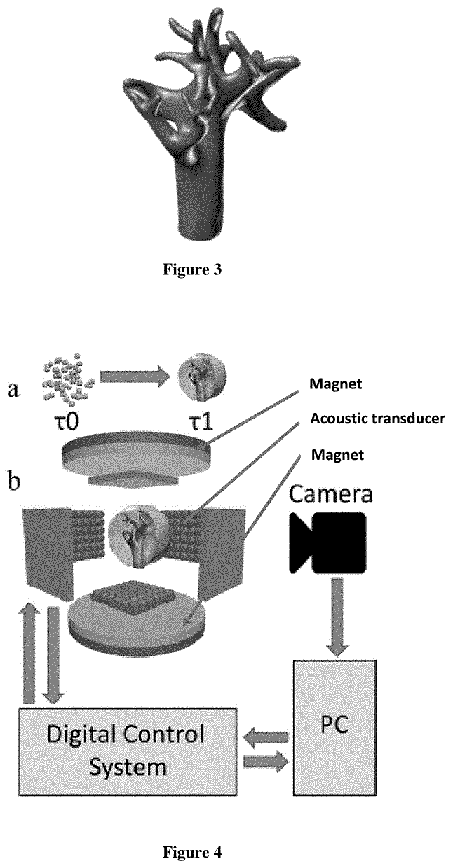

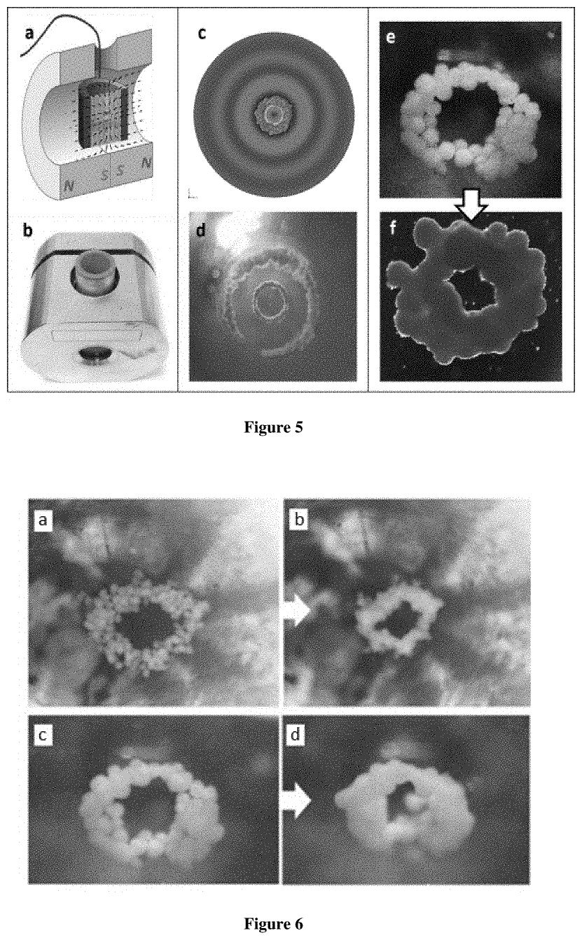

### TERMS AND DEFINITIONS

Definitions of some terms used in this description are given below. Unless otherwise specified, technical and scientific terms in this application have standard meanings commonly used in scientific and technical literature.

“Construct” means a continuous, i.e. fused, integral construct generated by means of magnetoacoustic fabrication.

In this context, the term “tissue spheroids” (or “spheroids”) relates to tissue spheroids which may be created from cells of different types. For example, spheroids may consist of, but not limited to, fibroblasts, chondrocytes, keratinocytes, primary astrocytes, thyrocytes, MMSCs (multipotent mesenchymal stromal cells), tumoral line cells (e.g., human melanoma cells). In some embodiments of the method different types of tissue spheroids (i.e. consisting of different types of cells) may be used for simultaneous fabrication. According to the invention, tissue spheroids are used for creation of tissue construction with maximum cellular density. “Tissue spheroids” represent small spherical pieces of tissue consisting of 2,000 to 3,000 cells. Tissue spheroids are convenient in work due to their sensible size (˜0.2 mm), three-dimensional spherical structure and fusion capacity which is permanent feature of any living tissue.

In this context, the term “medium” (“nutrient medium”) means any medium intended for biofabrication.

For example, alpha-MEM medium for tissue spheroids consisting of keratinocytes, primary astrocytes and human melanoma cells may be used as a nutrient medium. DMEM medium for tissue spheroids consisting of fibroblasts, chondrocytes, MMSCs and tumor line cells may be used as a nutrient medium. F-12 medium or tissue spheroids consisting of thyrocytes, Chinese hamster ovary cell cultures and hybridome cells may be used as a nutrient medium. RPMI-1640 medium for tissue spheroids consisting of lymphoid cells may be used as a nutrient medium. DMEM/F12 medium for tissue spheroids consisting of pancreas gland cells may be used as a nutrient medium.

“Paramagnetic medium” (“paramagnetic nutrient medium”) means a medium for biofabrication containing paramagnetic substance. Any compounds having paramagnetic properties, i.e. those obtaining magnetization directed along magnetic field intensity vector when placed in external magnetic field, may be used as paramagnetic substances. According to the invention, the most preferable paramagnetic substances are those having no toxic effect on cultured material, such as gadolinium salts and chelates, copper sulfate, manganese chloride, etc. Minimum concentration of paramagnetic substance is chosen which allows to provide material levitation in a non-uniform magnetic field. This concentration depends on material type, magnetic field parameters, medium composition and other biofabrication conditions (temperature, etc.). For example, when using Gd³⁺ gadolinium salts as paramagnetic substance its concentration may be 0.1 to 5,000 mM depending on selected material. In particular embodiments of the invention, gadolinium concentration may be 0.1 to 50 mM in order to obtain constructs from tissue spheroids consisting of different cell types.

In this context, the term “magnetic trap” means spatial configuration of the magnetic field created to limit any object movement. According to the invention, the “magnetic trap” appears in a central area of the non-uniform magnetic field and is characterized by the increase of field density when the object is removed from the magnetic trap in any direction. The “magnetic trap” is characterized by the lowest magnetic field density parameters ensuring transfer and subsequent assembly of levitating material in the magnetic trap.

In the present description and the claims of the invention, the terms “includes”, “comprises” and other grammatical forms shall not be interpreted in sole meaning but shall be used in a non-exclusive meaning (i.e. “having smth. in its composition”). Only expressions similar to “consisting of” shall be considered as an exhaustive list.

### DETAILED DESCRIPTION OF THE INVENTION

This invention is aimed at a new method of levitation bioassembly of three-dimensional tissue constructions by means of hybrid magnetoacoustic field. While there is a growing list of published works on magnetic or acoustic bioassembly, this invention is the first to discover hybrid magnetoacoustic levitation assembly of functional human tissues.

It means that three-dimensional tissue constructs obtained as a result of such bioassembly are viable and contractive.

The following was fabricated and studied using a developed method: tubular three-dimensional tissue construction consisting of smooth muscle cells (SMCs) and human urinary bladder tissue samples, and circular structure consisting of smooth muscle cells acting as vessel wall cells and chondrocytes forming a cartilage.

Herewith these examples of the use of hybrid magnetoacoustic field for complex shape tissue constructs fabrication are not limiting. Construct's shape is defined by acoustic radiation force. As a result, tissue constructs of spherical, ellipsoidal, circular and other shape may be obtained just by choosing of a suitable acoustic field sources configuration.

The possibility of objective demonstration of technical result when using the invention is confirmed by reliable data given in examples containing experimental data obtained in the process of research according to the methods adopted in this field.

It should be understood, that the examples given in application materials are not limiting and are given only for illustration of the present invention.

### Example 1. Biofabrication of Circular Construct from Tissue Spheroids in Magnetoacoustic Field

Basic tool used for the assembly is a combination of non-uniform magnetic field and standing acoustic field acting on the test tube with medium creating magnetoacoustic trap in certain area.

The apparatus layout is given in FIG. 5a. The experimental apparatus for creation of circular tissue construction consisted of two circular magnets joined together by opposite poles. Cylindrical cavity for piezoelectric transducer placement was made in the center of the apparatus. Cylinder-shaped piezoelectric transducer was used to put spheroids construction into a tubular shape such as circle or tube. Cylindrical ultrasonic transducer was placed in a hollow space between magnets and assembly working area (medium with tissue spheroids) was located in a piezoceramic cylinder (FIG. 5b).

The magnetic part of the experimental apparatus creates a local minimum of magnetic field potential. Due to magnetic field non-uniformity and the fact that relative permeability of spheroids differs from permeability of background fluid the magnetophoretic force appears. This causes particles movement to the areas with low magnetic field potential.

Standing acoustic field close to cylindrical field was created in the cavity within the transducer, therefore, field nodes had a cylindrical shape as well. Spheroids falling into such field nodes formed a tubular construct. Tubular structure walls thickness was defined by spheroids size and acoustic field amplitude and its length was defined by spheroids quantity. Tubular structure depended on wavelength radiated by acoustic transducer. Due to a small size of the constructions, manipulation with tissue spheroids in nutrient fluid is possible in ultrasonic frequency range—from hundreds of kilohertz to several megahertz. Ultrasonic wave frequency was chosen basing on the following two conditions: it shall be close to resonance for both transducer thickness and standing cylindrical wave in piezoceramic acoustic transducer cylinder.

Experimental Apparatus.

In more detail, acoustic transducer in the form of piezoelectric cylinder with inner radius Rᵢₙ=16 mm, outer radius of 22 mm and height of 20 mm was used as an acoustic field source. Piezoceramic material LITC-4 (FIG. 5b) was used as a transducer material. Wall thickness of 2 mm conformed to thickness resonance frequency of 770 kHz. In order to create standing wave within a transducer, radiation frequency should have been chosen which would conform to geometric resonance of standing cylindrical wave, i.e. would be defined by condition: J₀(kRᵢₙ)=0, where k=2πf/c—wave number, J₀—zero-order Bessel function. At the same time, radiation frequency shall be close to thickness resonance frequency for efficient electroacoustic transformation. In experimental conditions it was necessary to radiate ultrasonic wave with a frequency of 780 kHz to obtain a tubular structure of 1.5 mm in diameter.

Spheroids were placed in nutrient solution optimal for chosen cells type. Smooth muscle cells and chondrocytes were used in the experiment. The experimental apparatus was placed in thermostat which kept a temperature of 37° C. Gadolinium (paramagnetic substance) salts in concentration of 50 mmole/mole were dissolved in nutrient medium to perform magnetic levitation.

Numerical Assembly Simulation: Magnetic and Acoustic Field Calculation.

Numerical simulation was performed to predict construction assembly result. Simulation of a three-dimensional non-uniform static magnetic field in paramagnetic medium made of two permanent magnets was performed by the finite elements method. Ultrasonic field was calculated in accordance with a found field of displacements created by piezoelectric transducer using COMSOL computational software.

Gorkov's potential (acoustic radiation force potential) was used for acoustic radiation force calculation. Such approach represents a quite good approximation for the case when the wavelength is much larger than the scatterer size [15]. Potential minimum conforms to the area to which radiation pressure forces are directed and where spheroids will be concentrated. Gorkov's potential gradient conforms to radiation force acting on spheroids. Since the field within the transducer has a concentric structure just as the radiation force field, then not one tubular structure of spheroids but several interleaved concentric structures may be formed in case of a strong acoustic field. However, the magnetic force has a horizontal component attracting spheroids to the center of the magnetic apparatus. So the amplitude of the radiated ultrasonic wave was chosen empirically basing on two requirements: it shall be higher than the magnetic force in the center of working area to form a circle, i.e. push spheroids from the center, but it shall be lower than the magnetic force in the field of second minimum of radiation force potential to involve all spheroids from working volume in single circle formation.

Physical characteristics of simulated particles were chosen in accordance with experimental measurements [16].

Results.

In both computer simulation and experiments tissue spheroids were gathered in standing ultrasonic field nodes while levitating in nutrient medium. Numerical simulation has shown than the highest acoustic radiation force acts in the center of piezoelectric transducer due to high Gorkov's potential gradients in this area. It means that the diameter of obtained tissue circle depends on the first node radius (FIG. 5c).

The radius of obtained construction conformed to the design radius of the first node from the center: it may be defined from the ratio r₁/λ=0.3827. For radiated frequency of 780 kHz at medium temperature of 37° C. conforming to sound velocity in liquid of 1,530 m/s, the design radius of the construction would be 0.74 mm.

The calculated value approximately conformed to construction diameter of 1.5 mm observed in the experiment in the same conditions.

By changing radiated ultrasonic wave frequency it was possible to control spheroids conglomerate diameter. Furthermore, if there were a lot of tissue spheroids with large working volume conforming thereto, then a second circle was formed which conformed to the second node of the standing wave (FIG. 5d).

After spheroids assembly in circular structure it was necessary to retain them in this condition for fusion and formation of continuous tissue construct. It's important to notice that ultrasonic wave intensity was sufficiently low to avoid tissue spheroids damage even during such long-term exposure. Circle configuration retention during 18 hours led to spheroids fusion (FIG. 5e-d) indicating that spheroids remained viable.

During the experiment the biofabrication of circular structures consisting of smooth muscle cells (FIG. 6a, b) acting as vessel wall cells and chondrocytes (FIG. 6c, d) forming a cartilage was performed. As the final result for both cell types, spheroids fused and formed a continuous tissue circle within approximately 20 hours indicating that the cells remained alive throughout the fusion process.

Therefore, tissue spheroids were assembled into continuous tissue construct using a combination of magnetic and acoustic fields in levitation condition directly in nutrient medium. The size of obtained construct corresponded to the design size, the possibility of manipulation with spheroids using ultrasonic wave to give a desired size to resulting structure was demonstrated. The proposed approach to the use of physical fields for creation of tissue structures having different shapes and functionalities is a new step in biotechnology and three-dimensional biofabrication development.

### Example 2. Biofabrication of Functional Tubular Construction from Tissue Spheroids Using Directed Magnetoacoustic Levitation Assembly

In this study a viable tubular construction was biofabricated from spheroids consisting of human urinary bladder smooth muscle cells by combination of magnetic levitation and acoustic assembly. Obtained construction consisted from three layers of spheroids, which, in theory, can form three layers of muscles of mature urinary bladder wall—external longitudinal layer, middle circular layer and internal inclined layer—if appropriate conditions for its maturation after assembly will be created.

In the future it is planned to add spheroids from urogenital system epithelial cells in order to simulate a structure of natural urinary system walls. Although the experiment shown below describes the creation of tissue construction from human urinary bladder smooth muscle cells (hbSMCc), it should be assumed that this approach may be applied to other tubular structures such as blood vessels, large bowel and trachea.

The following aspects were carried out in this study:

1. For biofabrication of tubular constructions and, in theory, more complex geometric shapes using acoustic field, agarous cuvette was created, inside which a medium with tissue spheroids was placed and which was placed in apparatus for hybrid magnetoacoustic field creation.

2. Furthermore, it is unlikely that tissue construction assembly in the magnetoacoustic field would be possible without preliminary mathematical simulation which allowed to predict assembly speed and optimize the range of experimental conditions in situation with a quite high consumption of resources, mainly electric power, thanks to unique magnetic infrastructure provided by European Magnetic Field Laboratory (EMFL).

3. In addition to viability and morphological characteristics evaluation, the most important feature of tubular constructions assembled from smooth muscle cells (SMCs) which shall be assessed and confirmed is their functional activity. Therefore, in this study we evaluated the ability of obtained tissue engineered tubular construction to contract in the presence of vascular narrowings or relax in the presence of vasodilator. In order to evaluate viability and functional contractive activity of obtained tissue engineered constructions assembled in a strong magnetic field using hybrid magnetoacoustic technology, the test with gadobutrol (for viability evaluation) and with endothelin-1 (for functional contractility evaluation) was carried out.

Magnetoacoustic Apparatus.

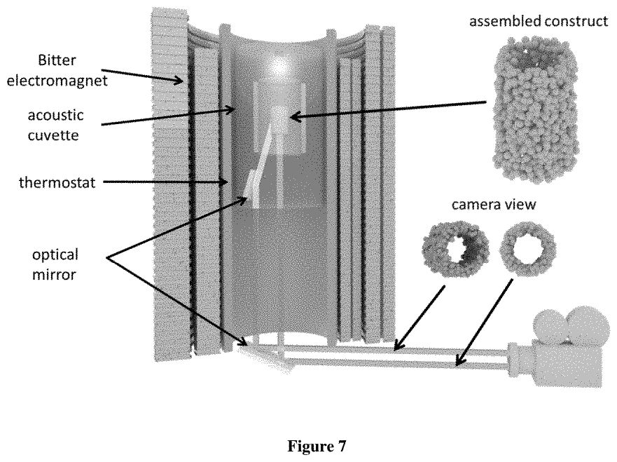

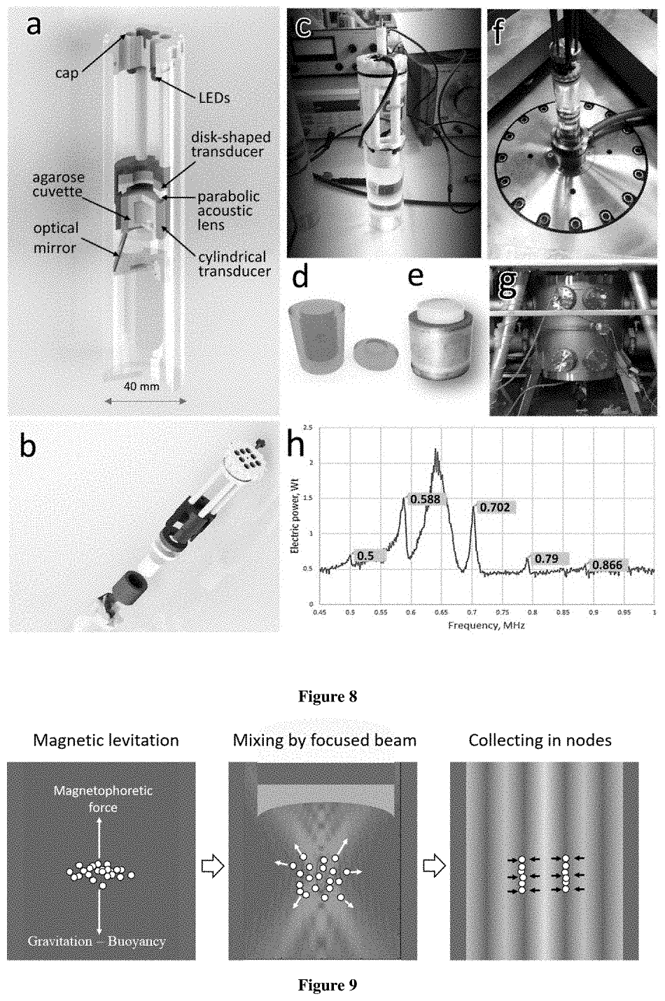

In order to provide magnetic levitation we used Bitter magnet with 50 mm hole and field density of 31 Tesla (FIG. 7) [17]. The method of assembly process control using mirrors system and digital camera is shown in FIG. 7. For biotechnology process a cuvette containing cylindrical and circular piezoceramic ultrasonic transducers, light-emitting diodes, optical mirror for monitoring, and agarous container for tissue spheroids (FIG. 8a,b) were used. Detailed description of experimental apparatus and cuvette design will be provided below.

the piezoceramic cylindrical acoustic transducer (inner diameter 8 mm, wall thickness 2 mm) generated standing ultrasonic waves at the frequencies providing tissue spheroids assembly in circular and tubular tissue constructions.

The circular transducer in combination with focusing parabolic plate located above the cylindrical acoustic transducer and focused inside a hollow space of cylindrical acoustic transducer was used to provide tissue spheroids mixing before assembly process start (FIG. 9). Each transducer is switched on and off successively: at first, circular transducer is switched on for spheroids mixing, then it is switched off and the cylindrical transducer is switched on for tubular structure formation.

The agarous container with tissue spheroids and culture medium containing 20 mM of gadobutrol (FIG. 8d) was placed inside the cylindrical transducer as shown in FIG. 8e. Agarose density and sound velocity are very close to values for the water, so the use of the agarous container instead of conventional plastic or glass was necessary to avoid additional waves reflection and distortion. Assembled cuvette was placed inside Bitter magnet (FIG. 8f, g) with magnet field density of ˜9.5 T. Formation of circular and tubular structures with different diameters was achieved due to the application of acoustic waves with several resonance frequencies in the range of 0.5 to 1 MHz. Rated acoustic wave amplitude at generator output was up to 10 V.

In order to create the most efficient standing wave using the cylindrical transducer it was necessary to create system resonance and, at the same time, to achieve maximum power output. Transducer placement inside a cuvette as well as addition of culture bottle and reflector in the system can change electric power function of frequency and shift resonances. Frequency dependence of transducer electric power was measured to set maximum power parameters. It was found that the maximum of radiated power of the transducer conforms to frequency of 0.64 MHz, and a number of secondary resonances exists (FIG. 8h). This frequency assembly was connected with different configurations of permanent ultrasonic field and allowed creating tubular constructions with different diameters.

After assembly the tissue construction was held in supporting magnetoacoustic field for 8 hours to complete tissue spheroids fusion process. Upon biofabrication process completion the obtained tissue construction was carefully transferred from the magnetoacoustic apparatus to culture dishes fur subsequent functionality testing and histological analysis.

Description of Cuvette Design Used in the Magnetoacoustic Apparatus.

Since the area inside Bitter magnet where a strong magnetic field is created has a diameter of 50 mm and an active core length of 1 m, special equipment was developed for experiment on magnetoacoustic particles assembly.

An acoustic unit was inserted directly in thermostat hole having a diameter of 4 cm. The thermostat was made in the form of hermetically sealed transparent cylindrical cuvette made of Plexiglas with ZEDEX plastic cover. Materials for cuvette and cover were selected from among materials with low porosity since the appearance of bubbles in ultrasonic radiation area is undesirable. As such, they may cause an additional acoustic wave attenuation and resonate, and also prevent visual observation of experiment.

A platform with vertically located cylindrical piezoelectric transducer was attached to the cover on plastic rods. The wires connected with transducer and passing through the center of the cuvette were firmly attached to the cover and put out for subsequent connection to signal generator and oscilloscope. Furthermore, light emitting diodes connected to direct current source were attached to the cover on the side facing inside the cuvette.

In order to observe particles movement during the experiment we used optical scheme consisting of the mirror located at the angle of 45 degrees to vertical axis coinciding with vertical axis of Bitter magnet, focusing lens and video camera with aperture taking account of focus. Since the lower part of cuvette was transparent, this scheme allowed observation of construct assembly in vertical direction (FIG. 7). The mirror was located directly above Bitter magnet and video camera and focusing lens were located so that to observe construct assembly process.

The optical scheme installed inside the cuvette, directly under the piezoelectric transducer was used for assembled construction length evaluation. The small mirror was attached to a plastic ring firmly attached inside the cylindrical cuvette at a shallower angle to vertical line so that receiving video camera captured images through the mirror with shifted angle of 45 degrees.

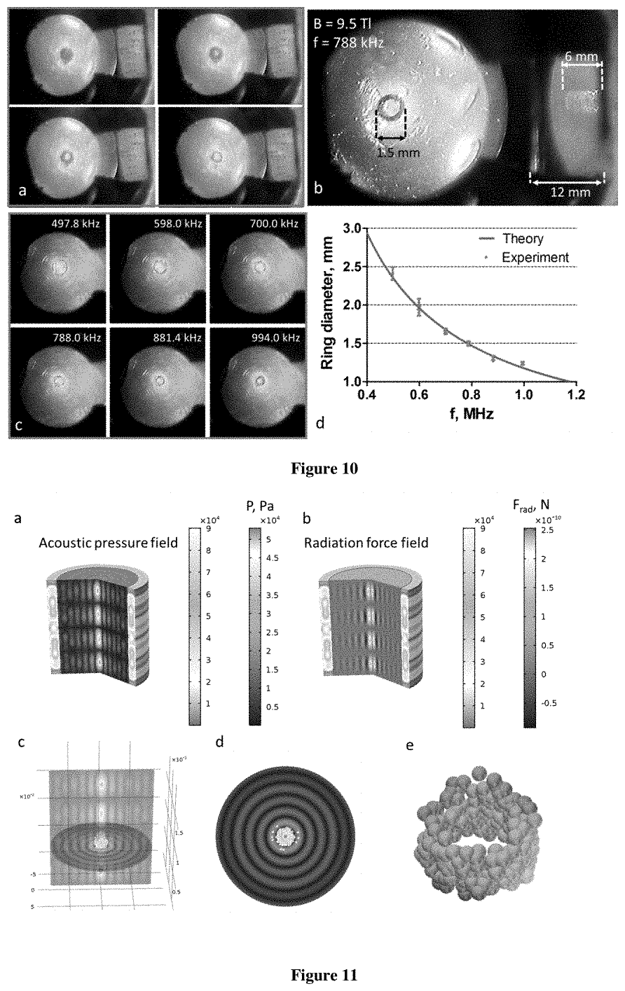

The system also includes additional horizontally oriented mirrors, which reflect the light impinging on particles from above for better contrast of obtained image. Therefore, bottom and side view of the construction were obtained during experiment (FIG. 10b).

Interior volume of the cuvette was filled with aqueous solution. Firstly, the presence of water ensures the absence of air layers between transducer wall and vessel with particles, which increases ultrasonic exposure efficiency. Secondly, the presence of significant water volume ensures more stable temperature conditions inside the transducer during ultrasonic radiation and protects particles against overheat or rapid cooling after withdrawal from thermostat. Thirdly, the distance between upper and lower butt ends of cuvette and cylindrical transducer allowed to decrease their contribution to resulting ultrasonic field distortion.

In order to assemble a three-dimensional construct the agarous container for particles collection having the shape of cylinder with tapered hole tightly closed with agarous cover. The container was shaped as follows: heated and melted agarose was poured in a special silicon mold of a desired shape. After cooling down to room temperature and curing, agarose was denested and used in the experiment.

A solution with paramagnetic gadobutrol salt was placed inside the agarous cuvette. This solution also contained polystyrene balls or tissue spheroids. Polystyrene balls had a size of 275 μm. The use of the agarous cuvette instead of a standard plastic cuvette was caused by several reasons. Firstly, the presence of solid walls in plastic cuvette generates additional reflection and absorption of ultrasonic wave. In case of imperfect coincidence of cuvette center with cylindrical transducer center, resonance conditions are disturbed and radiation power decreases.

At the same time, physical properties of the agarous container are very close to those of the water, therefore, container walls do not generate strong reflection and absorption of ultrasonic waves. The acoustic transparency of agarose excludes the necessity of container alignment with cylindrical piezoelectric transducer while the optical transparency allows observing experimental processes using a video camera. Secondly, plastic cuvette bottom is adhesive for fabricated spheroids and polystyrene balls, so they do not adhere to container bottom.

The container was placed in cylindrical piezoelectric transducer, which, in its turn, was attached to cuvette cover with plastic rods of predefined length. Using this rod allows lowering a platform with attached cylindrical and circular transducer and the container with particles randomly distributed in medium to desired height inside Bitter magnet. The length of the rods is chosen to place the cuvette in the area of the lowest magnetic field density, therefore, it depends on induction values of the magnetic field created inside Bitter magnet. Particles in this area do not levitate randomly under the action of the magnetic field but move slowly and approach each other.

Numerical Assembly Simulation: Calculation of Magnetic Field, Acoustic Field and Particles Movement Dynamics.

The experimental apparatus design was based on numerical simulation results. Such evaluations were necessary for definition of acoustic pressure distribution and simulation of acoustic projector and magnetophoretic forces action. The evaluation of optimal experiment parameters was performed by finite elements method using COMSOL Multiphysics and Matlab software.

Cylindrical piezoelectric transducer with radial polarization created the acoustic field with the internal and external radii of 8 mm and 10 mm, respectively, and the length of 20 mm. The distribution of acoustic pressure created by cylindrical piezoelectric transducer in the area of interest and its action on particles in shown in FIG. 11, a, b.

In the experiment, spheroids were localized in internal area of the cylindrical piezoelectric transducer, therefore, it was necessary to obtain a high level of field uniformity in vertical direction. However, the actual field structure was not completely uniform: surface acoustic waves necessarily appear at transducer-to-liquid interface, and this fact, in its turn, even in the case of small transducer surface defects leads to acoustic pressure amplitude change [18].

Gorkov's potential was calculated based on obtained distribution of resulting acoustic pressure field, and the radiation force acting on the spherical particle placed in acoustic field was determined basing on this potential [19]. Dummy particles' properties conformed to physical characteristics of real tissue spheroids (ultrasound velocity was 1,600 m/s, as for muscular tissue, density was 1,050 kg/m³, and diameter was 0.2 mm).

Follow the link https://www.hfml.ru.nl/luong/cal_cell.htm to see the magnetic field distribution. The magnetic field changed along the axis and remained uniform in horizontal plane. The following parameters were used for magnetophoretic force calculation: relative magnetic permeability of spheroids and medium was μ_sph=0.999992 and μ_f=0.9999994, respectively, and magnetic field density was 9.5 T.

In order to predict the particles movement trajectory in the magnetoacoustic field and the solid tube structure formation, the movement of non-stationary particles exposed to all acting forces (acoustic radiation force, magnetophoretic force, Stokes resistance force, elastic force of particles interaction and force of gravity) was simulated. As expected, the particles gathered in standing acoustic waves' nodes levitated under the action of magnetophoretic force and formed a solid tube with a radius equal to the radius of the first permanent field node. The results of numerical simulation (FIG. 11c, d, e) were reproduced experimentally. The shape of fabricated tissue construction is in good agreement with simulation results.

Assembly of Three-Dimensional Tissue Constructions in High Intensity Magnetic and Acoustic Fields.

Tissue spheroids levitation in a strong magnetic field with intensity of 9.5 T was shown. After the acoustic field generation the levitating tissue spheroids began gathering in circular and tubular structures (FIG. 9).

FIG. 10 a shows transformation of randomly distributed particles in a circle by step-by-step adjustment of acoustic wave amplitude. The construction height depended on the number of particles initially placed in the cuvette. FIG. 10b shows the obtained tubular construction (bottom and side view through mirrors system) observed at several frequencies. Gradual change of resonance frequency has led to shift in circular construction diameter (FIG. 10c). Thus, it is possible to adjust the assembly parameters to obtain a desired construction size. The dependence of the assembly diameter on frequency was measured experimentally (FIG. 10d) and was in good agreement with theoretical evaluation.

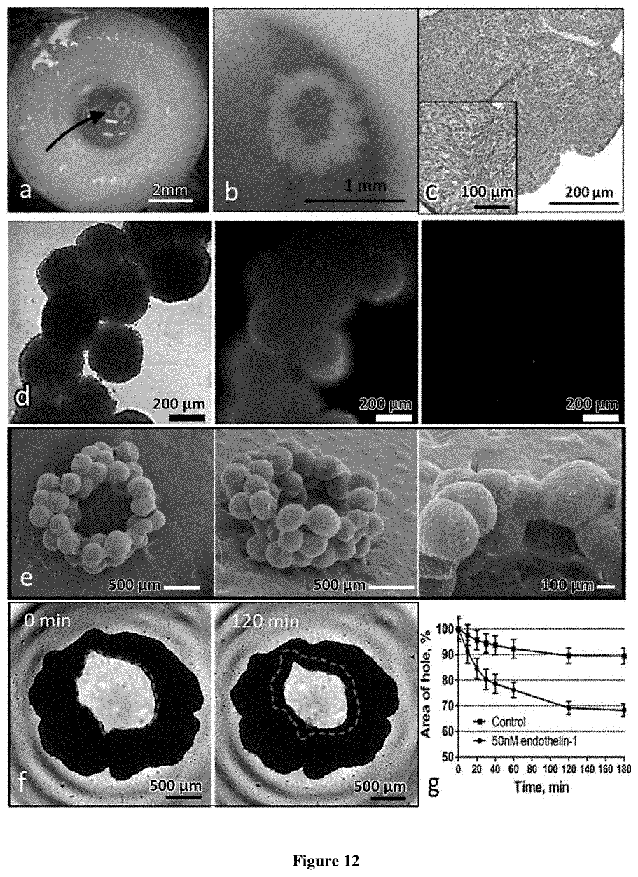

FIG. 12a, b shows the tubular tissue construction created from tissue spheroids after 8 hours of magnetoacoustic levitation assembly. Minor changes in tubular tissue size (wall thickness increase) occur within 8 hours but this shall not be reflected on a stable retention thereof by the acoustic field.

Cell Culture.

Smooth muscle cells (SMCs) of human urinary bladder (named hereinafter as hbSMC) were acquired from ScienCell (cat. No. 4310) and cultured in a serum-free medium for SMC with growth additives (SMCM, cat. No. 1101, ScienCell, USA). The cells were incubated at 37° C. in humidified atmosphere with 5% of CO2 and routinely splitted at 85-95% confluence. Single-cell suspension was prepared using mild enzymaticdissociation with a 0.25% solution of trypsin/0.53 mM of EDTA (cat. No. P043p, Paneco, Russia). The cells were free of mycoplasmal contamination, which is confirmed by DAPI staining protocol (cat. No. D1306, Invitrogen, USA).

Tissue Spheroids Formation Using MicroTissues 3D Petri Dishes.

Tissue spheroids were routinely prepared using micromolds for MicroTissues 3D Petri dishes (Z764019-6EA, 81 round well 800 μm×800 μm, Sigma Aldrich, USA) in accordance with the production protocol. Briefly, hbSMC cells were harvested from culture flasks and then suspended in the cells culture medium at concentration of 6.8×106 cells per millimeter. Then 190 μL of suspension was placed in each 81-well nonadhesive agarous mold, and the molds were placed in 12-well cultural plates (Nunc, USA) and in an hour they were covered with complete growth medium. The obtained tissue spheroids contained 1.6×104 cells.

Analysis of Spheroidal Synthesis.

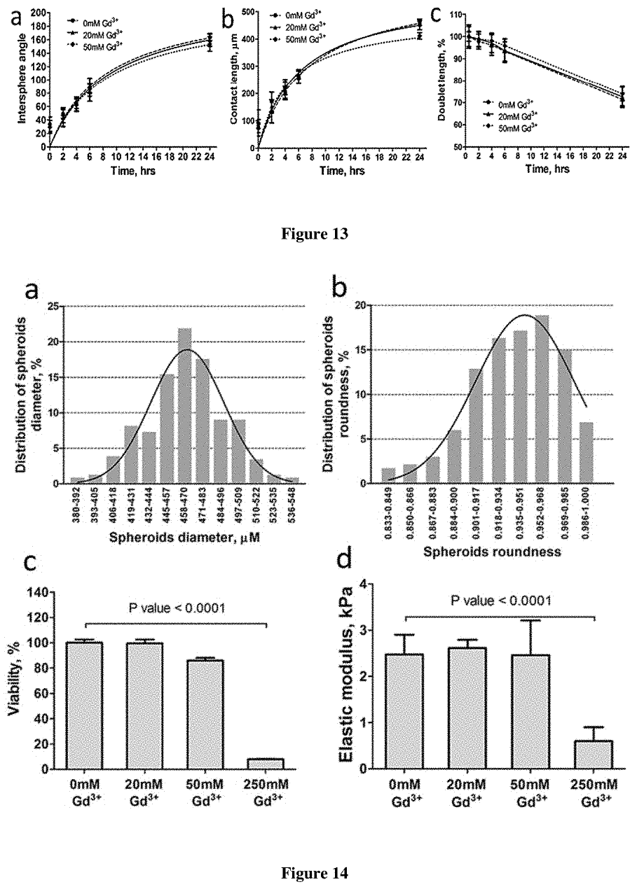

Tissue spheroids fusion dynamics in the presence of gadobutrol in different concentration during 24 hours (FIG. 13) was investigated.

Spheroidal fusion assay was performed using ultralow adhesive spheroidal microplates (cat. No. 4520, Corning, USA). Pairs of one-day tissue spheroids (16,000 cells per spheroids) were placed together in wells and incubated with 0.20 and 50 mM gadobutrol (Gd-DO3A-butrol, «Gadovist», Bayer Pharma AG, Germany) for 24 hours. Bright-field images of spheroidal doublets were obtained in points at 0, 2, 4, 6 and 24 hours using Nikon Eclipse Ti-S microscope. Contact length, intersphere angle and doublet length were measured using Image J 1.48v software (NIH, Bethesda, Md., USA) and plotted on the graph as time function using GraphPad Prism software (GraphPad Software, Inc., La Jolla, Calif.).

All pairs of spheroids demonstrated roughly the same fusion speed irrespective of the presence of gadobutrol. The contact length increased gradually as the time function and after 24 hours it was equal to initial diameter of a single spheroid. At the same time, the contact length growth for backup spheroids in 50 mM gadobutrol was little slower than the contact length growth for backup spheroids in 20 mM gadobutrol and without addition of gadobutrol. Intersphere angle increased to 160° indicating almost complete spheroidal fusion. The double length decreased successively and was equal to 72% of initial value after 24 hours of incubation.

Definition of Spheroid Diameter and Roundness Distribution

Tissue spheroids were biofabricated and captured on the first day using bright field visualization on inverted microscope Nikon Eclipse Ti-S, Japan. Spheroids diameter and roundness were measured using Image J 1.48v software (NIH, Bethesda, Md., USA). Brielfy, all original grayscale images were converted to simplified threshold images with similar conversion conditions, and the edges of spheroids were found automatically. MinFeret diameters of the exposed spheroid edges were initially measured as pixels and converted to micrometers by comparison with the reference length. The roundness was measured using Image J 1.48v shape descriptor.

Gadobutrol Influence of Tissue Spheroids Viability and Their Viability Evaluation at Different Gadobutrol Concentrations.

Tissue spheroids of correct shape and size were prepared using Petri micromolds MicroTissues 3D (FIG. 14a, b). The mean diameter of one-day spheroid was 454±25 μm. The mean spheroid roundness was 0.93±0.04.

First of all, gadobutrol influence of tissue spheroids viability was evaluated (FIG. 14c). Tissue spheroids viability was evaluated using a kit CellTiter-Glo 3D (cat. No. G9682, Promega, USA) based on bioluminescent ATF detection in viable cells in accordance with the manufacturer's protocol. One-day tissue spheroids (16,000 cells per spheroids) were exposed to 0, 20, 50 and 250 mM gadobutrol for 24 hours. Then the kit CellTiter-Glo 3D was added, and luminescence was recorded after 60 minutes of incubation using VICTOR X3 Multilabel Plate Reader (Perkin Elmer, USA). At 20 mM of gadobutrol tissue spheroids demonstrated almost 100% viability, while 50 mM gadobutrol caused viability decrease to 87% (FIG. 14c). Significant toxic effect on tissue spheroids was found and 250 mM of gadobutrol. It is worth noting that mechanical properties of tissue spheroids directly depend on their viability.

Gadobutrol Influence on Biomechanical Properties of Tissue Spheroids. Mechanical Testing.

Gadobutrol influence on mechanical properties of tissue spheroids was measured using tensometry by means of microscale parallel plates compaction testing system Microsquisher (CellScale, Canada) with suitable software SquisherJoy. Tissue spheroids (16,000 cells per spheroids) were prepared using Petri dish micromolds MicroTissues 3D. One-day tissue spheroids were exposed to 0, 20, 50 and 250 mM gadobutrol for 24 hours. Spheroids for mechanical testing were placed in a bath filled with phosphate-buffer saline (PBS) at 37° C. and compacted to 50% of strain for 20 seconds.

As shown in FIG. 14d, 20 mM and 50 mM gadobutrol did not change biomechanical behavior of spheroids in tissues, while gadobutrol strengthening to 250 mM lead to significant decrease of modulus of elasticity which was obviously caused by toxic effect of gadobutrol.

Living/Dead Cells (Kit).

Viability of cells in tubular construction fabricated from spheroids of hbSMC was controlled using the kit for living/dead cells staining (cat. No. 04511, Sigma-Aldrich, USA) in accordance with the manufacturer's protocol after 8 hours of incubation in a strong magnetic field. Tubular construction was incubated for 30 minutes with solution containing acetoxymethyl calcein (Calcein AM) and propidium iodide (PI) at 37° C. After washing with Dulbecco's phosphate-buffered saline (PBS, cat. No. 18912-014, Gibco, USA) tubular construction was visualized using fluorescent microscopy (Nikon Eclipse Ti-S, Japan).

Visualization results are shown in FIG. 12c, d. The construction consisted of viable cells' spheroids closely packed within the tissue. SEM analysis confirmed the fusion of tissue spheroids with tubular three-dimensional tissue constructions. As shown in FIG. 12e, the constructions consisted of three layers of spheroids. Spheroids' surface has a typical morphology of microspheres. It should be noted that incubation time in the magnetoacoustic field was insufficient for complete spheroids fusion, therefore contours of single spheroids were distinguishable. However, the constructions had a solid structure with a sufficient physical force for subsequent manipulations.

Histological Analysis.

After the assembly in a strong magnetic field the samples were fixed in 4% paraformaldehyde solution buffered with PBS (PFA, cat. No. P6148, Sigma-Aldrich) and then placed in melted 2% agarose gel (cat. No. Am-0710-0.1, Gelikon, Russia) and finally placed in paraffin (Merck, Germany). Xylene and a battery of downstream alcohol series were used for deparaffinization. Serial 4 μm thick slices obtained using Microtome Microm HM355S (Thermo Fisher Scientific, USA) were placed on a glass covered with poly-L-lysine and routinely stained with haematoxylin-eosin (Sigma-Aldrich, Germany).

Scanning Electronic Microscopy (SEM).

Tubular construct fabricated from hbSMC spheroids was fixed with phosphate-buffer saline (PBS) containing 2.5 v/v % glutaraldehyde (cat. No. G5882, Sigma-Aldrich, USA), dehydrated through ethanol series and dried using the critical point transition method on apparatus HCP-2 (Hitachi Koki Ltd, Japan). The sample was transferred to a metal plug with adhesive surface, covered with gold using ion sputtering source IB-3 (EIKO, Japan) and then observed using the microscope JSM-6510 LV (JEOL, Japan).

Tubular Constructions Contraction Analysis.

In order to evaluate the ability of hbSMCs in obtained three-dimensional tissue constructions to contract in response to addition of physiological vasoconstrictor, they were incubated with 50 nM of endothelin-1. Tubular construct was treated with 50 nM endothelin-1 (cat. No. E7764, Sigma-Aldrich, USA). The contraction of the construct was registered at points at 0, 10, 20, 30, 40, 60, 120 and 180 minutes using the Nikon Eclipse Ti-S microscope. The inner hole area of the tubular construction was measured using Image J 1.48v software (NIH, Bethesda, Md., USA) and plotted a function of time using GraphPad Prism software (GraphPad Software, Inc., La Jolla, Calif.). As shown in FIG. 12f, g, the agent caused a time-dependent inner hole area decrease indicating that the contractive response occurred. The decrease of the gap area is an indicative example for in-vitro model of a hollow organ with muscular wall.

In experimental conditions, the gap decreased significantly after addition of constrictor—to 70% of initial diameter, by 90% as compared to intact control. The greater part of area decrease occurred during first 120 minutes after addition of endothelin-1. Incubation during 60 minutes did not caused a subsequent contraction. Therefore, the functional activity of a hollow tubular tissue construction was demonstrated.

Data Analysis.

Statistical data were analyzed using GraphPad Prism software (GraphPad Software, Inc., La Jolla, Calif.) and presented as average value±S.E.M. Analysis of variance (ANOVA) test was used to determine significant differences between the average values of three and more groups with P-value <0.0001.

Conclusion.

Hybrid levitation magnetoacoustic biofabrication of three-dimensional functional tubular tissue constructions from hbSMC myospheres was carried out. The tubular construction fabricated from hbSMC responded to stimuli of endothelin-1 vasoconstrictor and was viable.

The experiments confirmed the concept of the use of solid bioassembly with magnetoacoustic levitation without frameworks, nozzles and tags for rapid fabrication of tissues and organ constructions with complex geometry. A subsequent scaling of technology and development of flow-type bioreactor systems will allow to create personalized implants conforming to anatomic and physiological characteristics of patient's organs for the purpose of clinical result enhancement. Generally speaking, the hybrid magnetoacoustic levitation bioassembly represents a new technology platform in a quickly developing field of forming biotechnological production.

Notwithstanding that the invention is described with the reference to embodiments disclosed, it should be obvious for professionals in the given field that the specific experiments detailed herein are only given for illustration of the present invention, should not be considered as limiting the scope of invention in any way. It should be understood that the implementation of different modifications is possible without diverting from the nature of the present invention.

### CITABLE DOCUMENTS

1. Elena A. Bulanova et al. Bioprinting of functional vascularized mouse thyroid gland construct.—Biofabrication 9(3). July 2017
2. I. Holland, J. Logan, J. Shi, C. McCormick, D. Liu, W. Shu, Bio-Design Manuf 2018, 1, 89.
3. V. Serpooshan, P. Chen, H. Wu, S. Lee, A. Sharma, D. A. Hu, S. Venkatraman, A. V. Ganesan, O. B. Usta, M. Yarmush, et al., Biomaterials. 2017, 131, 47.
4. Y. Zhu, V. Serpooshan, S. Wu, U. Demirci, P. Chen, S. Güven, Methods Mol. Biol. 2019, 1576, 301
5. T. Ren, P. Chen, L. Gu, M. G. Ogut, U. Demirci, Adv. Mater. 2020, 32, e1905713.
6. E. Türker, N. Demirçak, A. Arslan-Yildiz, Biomater. Sci. 2018, 6, 1745.
7. M. Anil-Inevi, S. Yaman, A. A. Yildiz, G. Mese, O. Yalcin-Ozuysal, H. C. Tekin, E. Ozcivici, Sci. Rep. 2018, 8, DOI 10.1038/s41598-018-25718-9.
8. S. Tasoglu, C. H. Yu, V. Liaudanskaya, S. Guven, C. Migliaresi, U. Demirci, Adv. Healthc. Mater. 2015, 4, 1469.
9. V. A. Parfenov, E. V Koudan, E. A. Bulanova, P. A. Karalkin, F. DAS Pereira, N. E. Norkin, A. D. Knyazeva, A. A. Gryadunova, O. F. Petrov, M. M. Vasiliev, et al., Biofabrication. 2018, 10, 034104.
10. W. L. Haisler, D. M. Timm, J. A. Gage, H. Tseng, T. C. Killian, G. R. Souza, Nat. Protoc. 2013, 8, 1940.
11. N. S. Lewis, E. El Lewis, M. Mullin, H. Wheadon, M. J. Dalby, C. C. Berry, J. Tissue Eng. 2017, 8, 2041731417704428.
12. A. Ito, K. Ino, M. Hayashida, T. Kobayashi, H. Matsunuma, H. Kagami, M. Ueda, H. Honda, Tissue Eng. 2005, 11, 1553.
13. M. Rogosnitzky, S. Branch, Biometals. 2016, 29, 365.
14. L. Ye, Z. Shi, H. Liu, X. Yang, K. Wang, J. Rare Earths. 2011, 29, 178.
15. A. V. Nikolaeva, et al., “Simulating and measuring the acoustic radiation force of a focused ultrasonic beam on elastic spheres in water”, Bulletin of the Russian Academy of Sciences: Physics 83, pp. 77-81 (2019).
16. E. V. Koudan, et al., “The scalable standardized biofabrication of tissue spheroids from different cell types using nonadhesive technology”, 3D Printing and Additive Manufacturing 4, pp. 53-60 (2017).
17. M. V. Berry, A. K. Geim, European Journal of Physics. 1997, 18, 307.
18. D. Cathignol, O. A. Sapozhnikov, J. Zhang, Journ. of Acoust. Soc. of America. 1997, 101, 1286
19. O. A. Sapozhnikov, M. R. Bailey, Journ. of Acoust. Soc. of America. 2013, 133, 661.

1. A method of three-dimensional tissue-engineered constructs fabrication from tissue spheroids randomly distributed in a working volume of medium which is paramagnetic relative to tissue spheroids placed in magneto-acoustic field representing a combination of non-uniform magnetic and acoustic fields, where magnetic field provides objects levitation due to field configuration with the lowest magnetic field density in the center of working volume of medium with tissue spheroids, and three-dimensional acoustic field forms internal and external construct geometry by means of acoustic radiation force.

2. The method according to the claim 1, wherein medium magnetic properties are provided by the presence of paramagnetic salts in the medium.

3. The method according to the claim 1, wherein magnetic field gradient in the direction of object force of gravity is at least 1.3 T/cm in order to ensure objects levitation.

4. The method according to the claim 1, wherein non-uniform magnetic field is generated using a magnetic system consisting of at least two circular permanent magnets with analogous poles facing each other.

5. The method according to the claim 1, wherein non-uniform magnetic field is generated using Bitter magnets.

6. The method according to the claim 4, wherein magnetic field intensity is equal to 2 T to 32 T.

7. The method according to the claim 1, wherein magnetic field is generated using superconducting magnets.

8. The method according to the claim1, wherein external geometry of three-dimensional tissue-engineered construct is chosen from: spheroidal, toroidal, ellipsoidal.

9. The method according to the claim 1, wherein internal and external geometry of three-dimensional tissue-engineered construct if formed by tissue spheroids exposure to at least one acoustic field structure which depends on acoustic source geometry, acoustic wave frequency and boundary conditions in exposure area.

10. The method according to the claim 9 wherein tissue spheroids exposure to different acoustic field structures is performed sequentially.

11. The method according to the claim 1, wherein acoustic field is a uniform field of standing ultrasonic wave.

12. The method according to the claim 1, wherein acoustic field is a non-uniform field representing a combination of standing and/or running ultrasonic waves propagating from one or more acoustic waves sources inclined angle-wise to each other.

13. The method according to the claim 1, wherein for the purpose of formation of internal construct geometry having the form of divided channels network within a construct, vector sum of acoustic radiation forces acting on tissue spheroids exceeds vector sum of other forces acting on tissue spheroids.

14. The method according to the claim 1, wherein three-dimensional tissue-engineered constructs fabrication is performed in three successive inseparable steps: construct assembly process, supporting stage and fusion stage.

15. The method according to the claim 14, wherein supporting stage lasts for 8 to 24 hours, and ultrasonic waves intensity is low to avoid tissue spheroids damage during long-term exposure.

16. The method according to the claim 14, wherein fusion stage lasts for 20 to 72 hours and is defined by the time necessary for generation of continuous tissue during tissue-engineered construct fabrication.

17. The method according to the claim 1, wherein external construct geometry is tubular.

18. The method according to the claim 17, wherein a cylinder-shaped piezoelectric transducer is used as acoustic waves source for acoustic field generation, a magnetic system in the form of two circular permanent magnets with analogous poles facing each other is used for magnetic field generation, where cylindrical piezoelectric transducer is installed in cylindrical gap of magnetic system.

19. The method according to the claim 18, wherein the working volume of medium with tissue spheroids is placed in an agarous container installed in the cylindrical piezoelectric transducer.

20. The method according to the claim 17, wherein a cylindrical piezoelectric transducer and a circular piezoelectric transducer with focusing parabolic lens are used for acoustic field generation, and a Bitter magnet is used for magnetic field generation.

21. The method according to the claim 19, wherein the working volume of medium with tissue spheroids is placed in the agarous container installed in the cylindrical piezoelectric transducer with the circular piezoelectric transducer with focusing parabolic lens located above it to focus ultrasonic wave in the cavity of the cylindrical piezoelectric transducer.

22. The method according to the claim 21, wherein piezoelectric transducers are located in a cylindrical thermostat which is located inside the Bitter magnet.
